(19) United States
(12) Patent Application Publication
(10) Pub. No.: US 2005/0228306 A1
(43) Pub. Date: Oct. 13, 2005

(54) System and method for filtering and detecting faint signals in noise

(75) Inventor: Isaac Kurtz, Toronto (CA)

Correspondence Address: BERESKIN AND PARR, 40 KING STREET WEST, BOX 401, TORONTO, ON, M5H 3Y2, CA

(73) Assignee: Vivosonic Inc., Toronto (CA)

(21) Appl. No.: 11/092,567

(22) Filed: Mar. 29, 2005

Related U.S. Application Data
(60) Provisional application No. 60/556,881, filed on Mar. 29, 2004.

(51) Int. Cl. A61B 5/04

(52) U.S. Cl. 600/544

(57) ABSTRACT

A system and method for filtering and detecting faint signals in noise by placing a filter prior to an amplifier. In particular, a system for filtering differential signals includes: a signal receiver for receiving a differential signal; a prefiltering network connected to the receiver for filtering the differential signal prior to amplification, the prefiltering network including a high-pass filter and/or a low-pass filter; and an amplifier connected to the filtering network. In contrast with the conventional approach of connecting the filter to the amplifier's inputs, the filtering network in a preferred embodiment of the invention is connected to the amplifier through the amplifier's gain resistor such that the input impedance and common mode rejection ratio of the amplifier is not adversely affected by the filtering. Band-pass filtering prior to amplification reduces the noise in the signal and allows higher gain to provide a clearer signal following amplification.

### FIELD OF INVENTION

This invention relates to the field of detecting faint electrical signals in noise and, more particularly, relates to a system and method for detecting evoked potential signals by filtering signals prior to amplification.

### BACKGROUND OF THE INVENTION

Evoked potentials (EPs) are very faint electrical signals that are produced within the body when subject to stimulus. EPs can be used, for example, in auditory testing. In particular, Auditory Steady State Responses (ASSR) are signals that are in the range of 10-50 nV (1 nV is a billionth of a Volt). Another type of EP is the Auditory Brainstem Response (ABR), which is in the range of 100-1000 nV.

When detecting EPs, the detection is affected by numerous types of electrical “noise” at the electrodes from external sources such as power lines, equipment in the area (including equipment used to stimulated the EP response), Radio Frequency interference and also from physiological sources, for example, from the brain (EEG), the heart (ECG), eyes (EOG), and muscles (EMG). FIG. 1 is a table listing types of EP signals and various types of noise that may affect the EP signals. FIG. 2 shows the voltages and frequency ranges for various types of noise.

Interestingly, the physiological sources of noise, such as ECG, can be more problematic in infants than adults because an infant's heart is positioned more centrally, is generally larger relative to the body, is closer to the head, and beats faster.

In a conventional EP detection system, electrode pads applied to the skin are connected to an amplifier through lead wires. The amplifier is then connected to an anolog to digital converter and then to a digital signal processor. The leads from the electrodes to the amplifier can also act like radio antennas that can pick up extraneous electrical and magnetic fields from surrounding equipment, lights, and the like. This effect is called electromagnetic interference (EMI). As an example, FIG. 3 illustrates the introduction of EMI to the EP signal.

As seen in FIG. 2, the various noise sources can have amplitudes that may be many hundreds of times larger than the ASSR signal. As such, in conventional systems, EPs are amplified with large gain and then filtered (often band-pass filtered (BPF)) to remove unwanted noise to represent the EP adequately for subsequent analog-to-digital (A/D) conversion and digital signal processing (DSP).

In a conventional amplifier, using a large gain can result in saturation (reaching the limits of the amplifier's dynamic range), which, after filtering using a band-pass filter, distorts the signal and can leave “blank” periods or pauses in the signal (as illustrated in FIG. 4). Conversely, using a lower gain reduces the signal-to-noise ratio (SNR) at the amplifier output, may require additional amplification and complicates signal detection in later processing.

Attempts to solve these problems include either: (1) placing low pass filtering elements in series with the instrumentation amplifier inputs; or (2) placing a high pass filtering element in a feedback path of the final stage of the amplifier circuit.

A disadvantage of the first approach is that the common mode rejection ratio (CMRR) and input impedance of the amplifier are adversely affected because small differences in components connected to the input leads cause the common mode gain to increase.

A disadvantage of the second approach is that the gain of the first stage of the instrumentation amplifier generally has to be limited to avoid saturation. Limiting the first stage gain adversely affects the SNR and the CMRR of the amplifier.

The following documents deal generally with filtering and/or detection of EP signals and are hereby incorporated herein by reference:
Identifying and Reducing Noise in Psychophysiological Recordings, Cutmore et al., Int. J. Physiol., V. 32, No. 2, May 1, 1999, pp. 129-150;
Input Filter prevents instrumentation-amp RF-rectification errors, Kitchin et al., EDN, Nov. 13, 2003, p. 101-102;
Suppression of low frequency effect of high frequency interference in bioelectrical recordings, deJager et al., 18^{th} Annual Conf. of IEEE Engineering in Medicine and Biology Society, Amsterdam; 1996, p. 26—;
AC-Coupled Front-End for Biopotential Measurements, Spinelli et al., IEEE Transactions on Biomedical Engineering, Vol. 50, No. 3, March 2003, p. 391—;
Scalp electrode impedance, infection risk, and EEG data quality, Ferree et al., Clin. Neurophysiol., 112, 2001, p. 536-544;
INA114 Precision Instrumentation Amplifier Data Sheet, Burr Brown Corporation, March 1998.

### SUMMARY OF THE INVENTION

The present invention is intended to overcome at least some of the noted issues. In one embodiment of the invention, there is provided a system and method to filter a differential signal in which band-pass filtering is performed before amplification without adversely affecting common mode rejection ratio and input impedance. The use of band-pass filtering prior to amplification reduces the noise in the signal being amplified and allows higher gain to provide a clearer signal following amplification.

According to an embodiment of the invention, there is provided a system for filtering differential signals that includes: a signal receiver for receiving a differential signal; a prefiltering network connected to the receiver for filtering the differential signal prior to amplification to reduce noise, the prefiltering network including a high-pass, low-pass, band reject or band-pass filter. The system may include an amplifier connected to the filtering network for receiving and amplifying the filtered signal.

In one aspect of the system, the differential signal may be a bioelectric signal and; more particularly, an evoked potential signal.

In another aspect of the system, the amplifier is an operational amplifier instrumentation amplifier and the prefiltering network is provided in the first stage of the instrumentation amplifier. In particular, the prefiltering network may be placed in series with the gain resistor of the instrumentation amplifier.

In various aspects of the system of this embodiment the prefiltering network may be designed, configured or arranged to reduce one or more of radio frequency interference, low-frequency noise, DC offset, or the like from the differential signal.

According to another embodiment of the invention, there is provided a system for detecting EP signals. The system includes: at least two active electrodes; a ground electrode connected to the at least two active electrodes for detecting the EP signal a prefiltering network provided in proximity to the ground electrode for receiving the EP signal and filtering noise from the EP signal; and an amplifier also provided in proximity to the ground electrode and connected to the prefiltering network to receive and amplify the EP signal.

In a particular case, the prefiltering network includes a high-pass, low-pass, band reject, or band-pass filter.

In another particular case, the amplifier may be an operational amplifier instrumentation amplifier and the prefiltering network is provided in the first stage of the instrumentation amplifier. In this case, the prefiltering network can be placed in series with the gain resistor of the instrumentation amplifier.

Similar to the above, in various aspects of the system of this embodiment the prefiltering network may be designed, configured or arranged to reduce one or more of radio frequency interference, low-frequency noise, DC offset, or the like from the differential signal.

According to yet another embodiment of the invention, there is provided a method for filtering differential evoked potential signals. The method includes: receiving a differential evoked potential signal; and filtering the signal to reduce noise using a prefiltering network prior to amplification, the prefiltering network including a high-pass, low-pass, band reject or band-pass filter.

Again in this method, the filtering can be designed to reduce one or more of radio frequency interference, low-frequency noise and DC offset from the differential evoked potential signal.

### BRIEF DESCRIPTION OF THE DRAWINGS

Features of the preferred embodiments of the invention will become more apparent with reference to the following detailed description in which reference is made to the appended drawings wherein:

FIG. 1 is a table listing types of EP signals and various types of noise which may affect the EP signals;

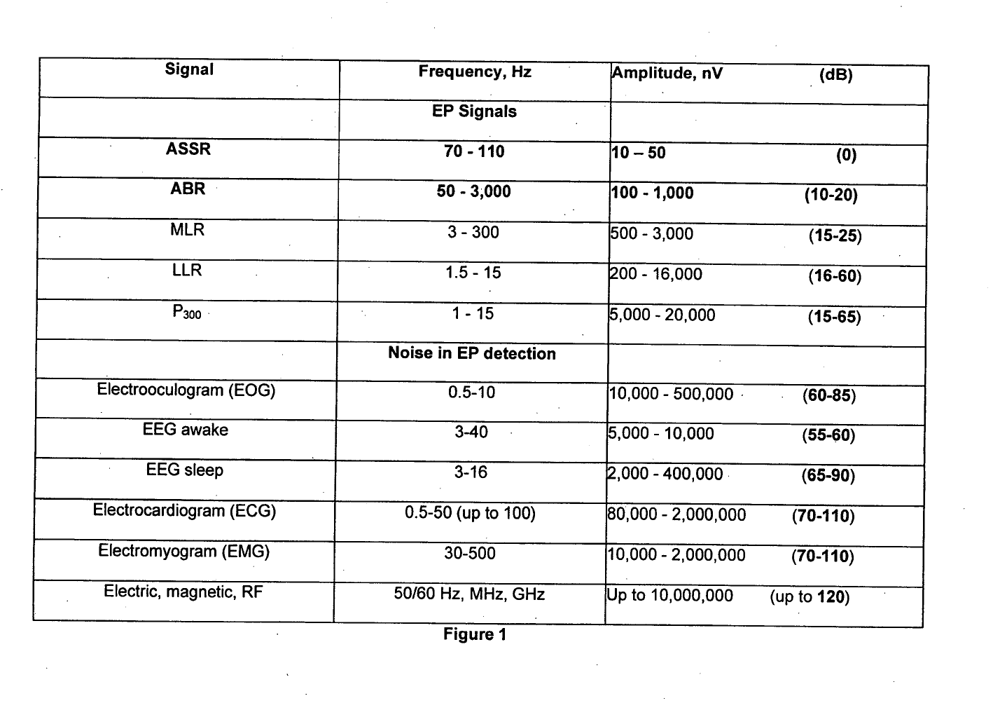

FIG. 2 shows the voltages and frequency ranges for various types of noise;

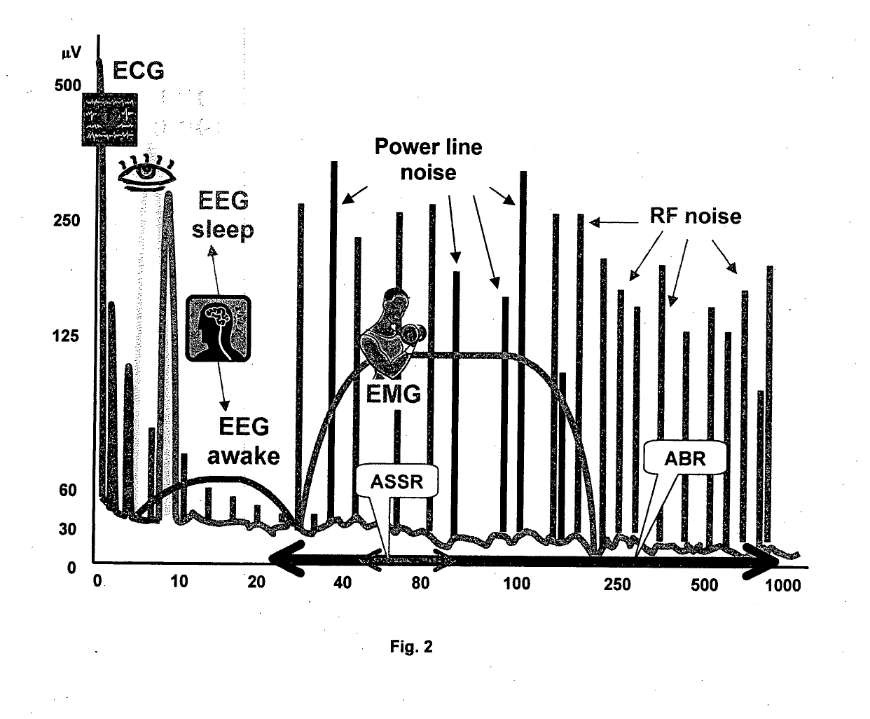

FIG. 3 illustrates the introduction of EMI in a conventional amplifier;

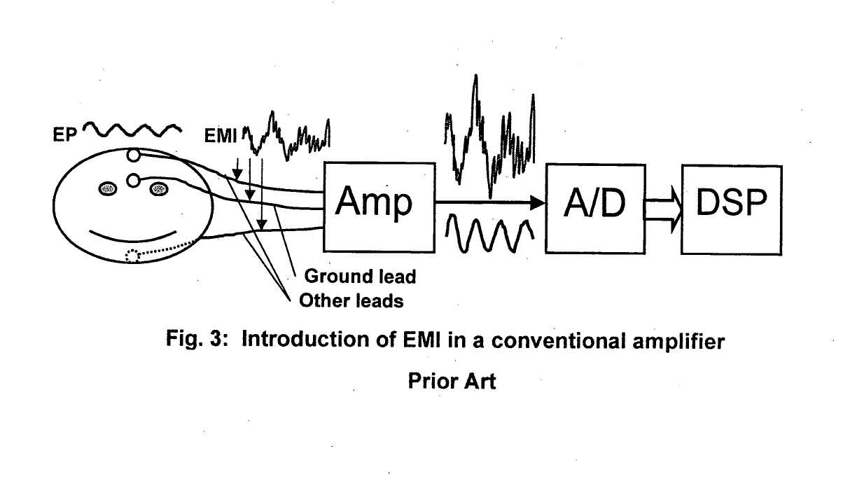

FIG. 4 illustrates signal distortion because of saturation in a conventional amplifier;

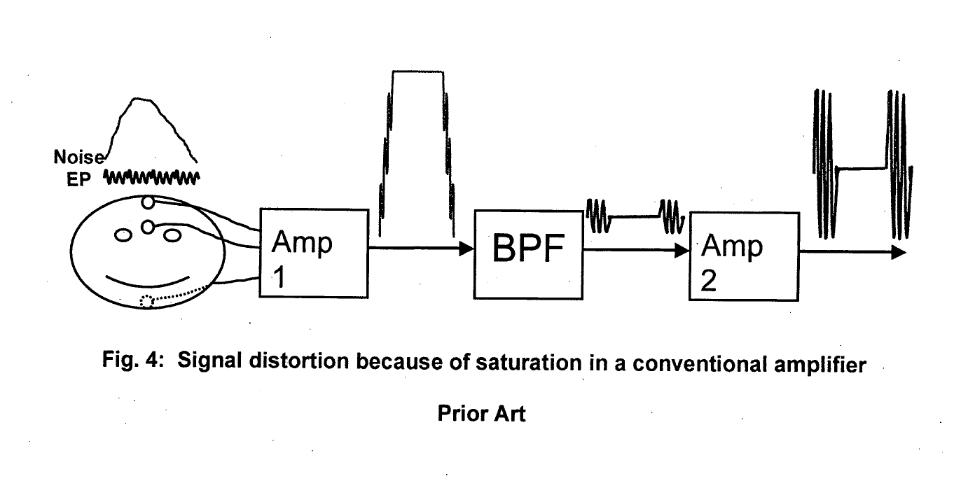

FIG. 5 is a schematic illustration of a system for detecting EP signals according to an embodiment of the invention;

FIG. 6 illustrates reduction of EMI using the embodiment of FIG. 5;

FIG. 7 shows a configuration of a prefiltering network and amplifier according to an embodiment of the invention; and

FIG. 8 illustrates high gain without distortion according to the embodiment of FIG. 7.

### DETAILED DESCRIPTION OF EMBODIMENTS

In an embodiment of the present invention a method of filtering signals prior to amplification includes filtering (low-pass, high-pass, band-reject or band-pass filtering) the signal prior to the first stage of amplification, thus reducing unwanted noise and allowing higher gain. As a result, EP signals at the output have larger amplitude, contain much less noise, and have larger SNR.

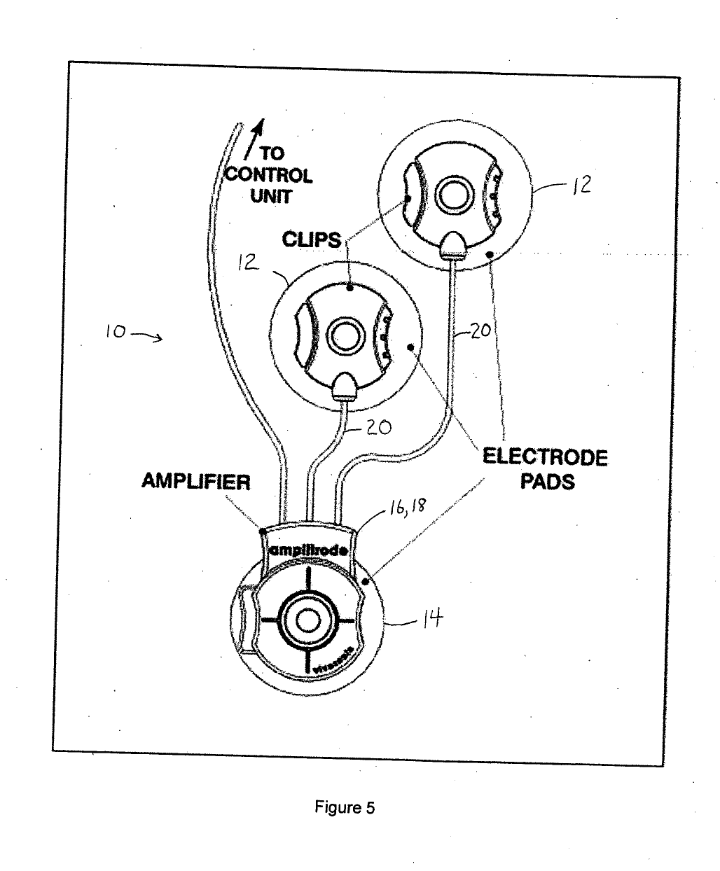

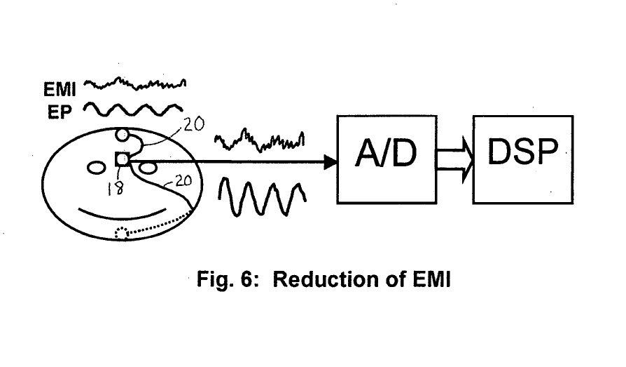

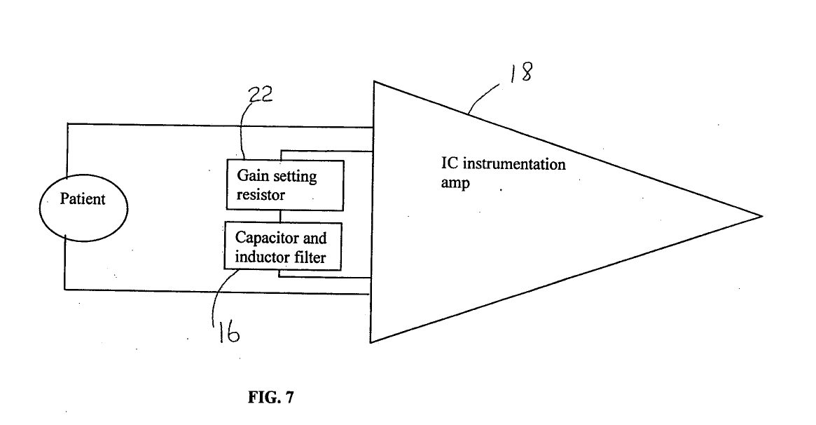

FIG. 5 shows a system 10 for measuring EP signals according to an embodiment of the invention. The system 10 includes two active electrodes 12 and a ground electrode 14 that allow the determination of a differential EP signal. In this particular embodiment, the ground electrode 14 includes a prefiltering network 16 and an amplifier 18 (shown in FIG. 7). The active electrodes connect to the prefiltering network 16. The provision of the prefiltering network 16 and amplifier 18 in proximity to the ground electrode 14 allows for shorter leads 20, which reduces EMI because the shorter leads are less likely to pick up the EMI. This effect is illustrated in FIG. 6.

FIG. 7 shows a configuration of the prefiltering network 16 and amplifier 18 according to an embodiment of the invention. The amplifier 18 is, for example, a three op-amp instrumentation amplifier, including a gain resistor 22. The three op-amp instrumentation amplifier 18 may be, for example, the INA114 Precision Instrumentation Amplifier from Texas Instruments Incorporated of Dallas, Tex. (formerly Burr-Brown Corporation) or similar. In this embodiment, the prefiltering network 16 is placed in series with the gain resistor 22 of the three op-amp instrumentation amplifier 18. In preferred embodiments, the prefiltering network 16 makes use of miniaturized passive electronic components, specifically inductors and capacitors. These electronic components are available with high values in small packages that allow these electronic components to provide a variety of filtering characteristics having a small overall circuit size. In a preferred embodiment, the amplifier 18, gain resistor 22 and prefiltering network 16 components could be included in a single integrated circuit package with appropriate specifications for EP amplification.

The prefiltering network 16 includes an appropriate high-pass, low-pass, band-reject, or band-pass filter, and preferrably a band-pass filter, to reduce the noise and allow for higher gain. As is known in the art, these types of filters comprise an appropriate arrangement of capacitors and inductors. It will be understood by one of skill in the art that inductor and capacitor values, as well as the inductor's series resistance, within the prefiltering network 16 will be chosen so as to appear as a minimum impedance in the frequency band of interest in order to maximize the amplifier gain. In particular, recent advances in the manufacture of passive components have led to very high valued inductors (10 mH) that are magnetically shielded and suitable for this embodiment. As an example, part number DS1608C106 from CoilCraft Inc. of Cary, Ill. may be used as an inductor.

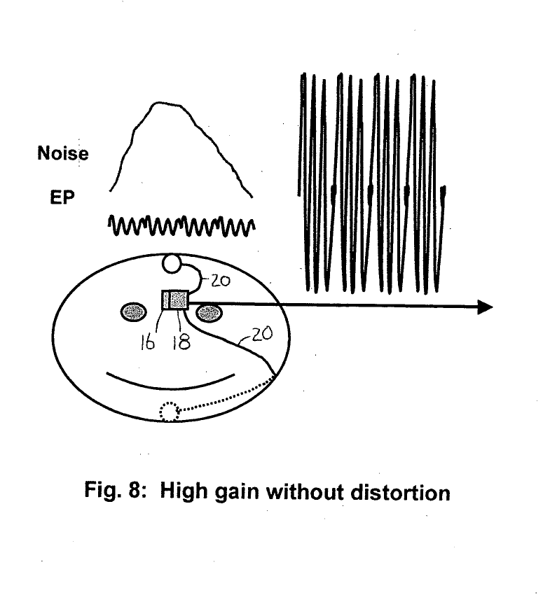

The use of a prefiltering network 16 in series with the gain resistor 22 overcomes the problems noted above that are associated with conventional techniques by reducing the various noise components in advance of the amplification stage. Thus, a cleaner signal is sent to the amplifier 18, which can then provide higher gain. This result is illustrated in FIG. 8.

In this embodiment, the prefiltering network is not connected to the amplifier inputs such that the prefiltering network has no effect on common mode rejection ratio or on amplifier input impedance. On the other hand, the signal filtering in this embodiment is also prior to the first stage of amplification, thus overcoming the problem of saturation.

It will be apparent to one of skill in the art that other combinations of known or hereafter known amplifiers and filtering networks may be used to achieve a similar result as the amplifier 18 and prefiltering network 16 described in the above.

Embodiments of the present invention provide band-pass (low-pass, high-pass, band-reject) filtering prior to amplification of any differential signal requiring amplification. In particular, the embodiments of the invention can be applied advantageously to bioelectric signals and evoked potential signals.

Although the invention has been described with reference to certain specific embodiments, various modifications thereof will be apparent to those skilled in the art without departing from the spirit and scope of the invention.

1. A system for filtering differential signals, said system comprising:
a signal receiver for receiving a differential signal;
a prefiltering network connected to said receiver for filtering said differential signal prior to amplification to reduce noise, said prefiltering network comprising:
a high-pass, low-pass, band-pass or band-reject filter; and
an amplifier connected to said filtering network for receiving and amplifying said filtered signal.

2. A system according to claim 1, wherein said differential signal is an evoked potential signal.

3. A system according to claim 1, wherein said differential signal is a bioelectric signal.

4. A system according to claim 1, wherein said amplifier is an operational amplifier instrumentation amplifier and said prefiltering network is in the first stage of said instrumentation amplifier.

5. A system according to claim 4, wherein said prefiltering network is in series with the gain resistor of said instrumentation amplifier.

6. A system according to claim 5, wherein said prefiltering network comprises a capacitor and an inductor.

7. A system according to claim 5, wherein said prefiltering network is designed to reduce radio frequency interference.

8. A system according to claim 5, wherein said prefiltering network is designed to reduce low-frequency noise.

9. A system according to claim 5, wherein said prefiltering network is designed to reduce DC offset from said differential signal.

10. A system for detecting EP signals, said system comprising:
at least two active electrodes;
a ground electrode connected to said at least two active electrodes for detecting said EP signal;
a prefiltering network provided in proximity to said ground electrode for receiving said EP signal and filtering noise from said EP signal; and
an amplifier also provided in proximity to said ground electrode and connected to said prefiltering network to receive and amplify said EP signal.

11. A system according to claim 10, said prefiltering network comprising:
a high-pass, low-pass, band-pass or band-reject filter.

12. A system according to claim 10, wherein said amplifier is an operational amplifier instrumentation amplifier and said prefiltering network is in the first stage of said instrumentation amplifier.

13. A system according to claim 12, wherein said prefiltering network is in series with the gain resistor of said instrumentation amplifier.

14. A system according to claim 13, wherein said prefiltering network comprises a capacitor and an inductor.

15. A system according to claim 13, wherein said prefiltering network is designed to reduce radio frequency interference.

16. A system according to claim 13, wherein said prefiltering network is designed to reduce low-frequency noise.

17. A system according to claim 13, wherein said prefiltering network is designed to reduce DC offset from said differential signal.

18. A method for filtering differential evoked potential signals, said method comprising:
receiving a differential evoked potential signal; and
filtering said signal to reduce noise using a prefiltering network prior to amplification, said prefiltering network comprising a band-pass filter.

19. A method according to claim 18, wherein said prefiltering network is in series with a gain resistor of an instrumentation amplifier.

20. A method according to claim 18, wherein said filtering is designed to reduce radio frequency interference, low-frequency noise and DC offset from said differential evoked potential signal.
